(19) United States
(12) Patent Application Publication
(10) Pub. No.: US 2023/0248876 A1
(43) Pub. Date: Aug. 10, 2023

(54) SELF-DISINFECTING ANTIVIRAL FILTER MATERIAL, MANUFACTURING AND USE THEREOF, AND AIR FILTER DEVICE WITH THE FILTER MATERIAL

(71) Applicant: FRAUNHOFER-GESELLSCHAFT ZUR FOERDERUNG DER ANGEWANDTEN FORSCHUNG E. V., Muenchen (DE)

(72) Inventors: Horst BOETTCHER, Dresden (DE); Michael HOFFMANN, Dresden (DE)

(21) Appl. No.: 18/015,660

(22) PCT Filed: Aug. 5, 2021
(86) PCT No.: PCT/EP2021/071855

§ 371 Date: Jan. 11, 2023

(30) Foreign Application Priority Data
Aug. 12, 2020 (DE) 102020121204.6

(51) Int. Cl. A61L 9/18; A41D 13/11; B01D 46/00; B01D 39/14; B01D 53/00; B01D 53/81; A61L 9/014; F24F 3/16; F24F 8/24; A62B 23/02

(52) U.S. Cl. CPC A61L 9/18; A41D 13/1192; B01D 46/0028; B01D 39/14; B01D 53/007; B01D 53/81; A61L 9/014; F24F 3/16; F24F 8/24; A62B 23/025; B01D 2279/65; B01D 2279/50; B01D 2239/0442; B01D 2239/0478; B01D 2239/10; B01D 2259/802; B01D 2259/4541; B01D 2257/91; B01D 2258/06; A61L 2209/14; A61L 2209/16

(57) ABSTRACT

A self-disinfecting antiviral filter material 10 includes an air-permeable carrier 11 and a composite coating 12 including at least one color pigment 13 with antiviral activity and an inorganic matrix material 14 bonded to the carrier 11. The at least one color pigment 13 preferably includes at least one compound from the group of phthalocyanines. The matrix material is preferably selected from the group including metal oxides. An air filter device including the self-disinfecting antiviral filter material, a method of manufacturing the filter material, and a method of inactivating viruses are also described.

The invention relates to a self-disinfecting antiviral filter material and an air filter device containing such filter material. The invention relates in particular to a filter material for breathing masks or air purification devices as used in air conditioning systems, for example. The invention also relates to a method of manufacturing the filter material and a method of inactivating viruses.

It is known to be very important in case of virus epidemics to provide protection against infection and the further spread of viruses by means of suitable air filter devices. In addition to a filtering effect, air filter devices should, if possible, also have antiviral and self-disinfecting effects. The wearing of flexible breathing masks or filters in particular is sensible or even necessary for personal protection. However, conventional filter materials known from practice have no or only a weak antiviral effect.

An advantageous, biocide-free method of achieving an antiviral effect is to use dyes that sensitize the formation of reactive, germicidal singlet oxygen through visible light and atmospheric oxygen (in its natural ground state as triplet oxygen), which is already used in medicine as so-called photodynamic therapy (PDT) to treat cancer. Phthalocyanines are of particular interest as suitable sensitizer dyes for use in filter materials, due to their broad absorption in the visible spectral range, their high light stability and their high quantum yields of singlet oxygen generation (see, for example, WO 2015/154543 A1). However, the use of phthalocyanines in filter materials has so far led to the following problems.

First, unsubstituted phthalocyanines are insoluble pigments in conventional solvents, making it impossible to coat the carrier from solution.

Second, the introduction of solubilizing cationic ammonium groups into phthalocyanines, as described in WO 2015/154543 A1 for the production of three-dimensional mask filters, has the disadvantage of slow diffusion (“bleeding”) of the water-soluble dyes in the presence of moist (breathing) air, in addition to the extremely complex synthesis. Much the same applies to the use of phthalocyanines containing solubilizing anionic sulphonic acid or carboxylic acid groups, as described in WO 2017/196961 A1.

Third, the grafting of phthalocya nine dyes containing reactive groups and forming a chemical linkage with the carrier requires a high process and synthesis effort and allows only limited use of special carriers that have appropriate docking sites (WO 2007/194843 A1).

Finally, the incorporation of metal phthalocyanines into polymers (see WO 2012/064894 A1) in principle allows coating without diffusion, but with very low singlet oxygen formation efficiency, since only the dye molecules lying on the polymer surface can react with atmospheric oxygen.

It is therefore the objective of the present invention to provide an improved filter material with an antiviral effect that avoids disadvantages of conventional filter materials. In particular, the antiviral filter material should be easy to produce by applying the antiviral component to different carriers, have high mechanical stability and light stability even over long periods of time, be self-sterilizing with high efficiency under room conditions (atmospheric oxygen, daylight or room light), and/or be suitable for use in air filter devices, in particular personal masks or equipment for cleaning room air. A further objective of the invention is to provide a correspondingly improved air filter device which is provided with the filter material and avoids disadvantages of conventional air filter devices. Other objectives of the invention consist in providing an improved method of manufacturing the filter material and an improved method of inactivating, in particular destroying, viruses.

These objectives are respectively solved by the filter material, the air filter device, the method of manufacturing the filter material and the method of inactivating viruses with the features of the independent claims. Preferred embodiments and applications of the invention are described in the dependent claims.

According to a first general aspect of the invention, the said objective is solved by a self-disinfecting antiviral filter material having an air-permeable carrier and a composite coating connected to the carrier and comprising at least one color pigment with antiviral activity and an inorganic matrix material.

According to a second general aspect of the invention, the said objective is solved by an air filter device, in particular a breathing mask or a room air filter, which is provided with a filter material according to the first general aspect of the invention.

According to a third general aspect of the invention, the said objective is solved by a method of manufacturing the filter material according to the invention or one of its embodiments, said method comprising at least the following steps. The carrier is provided. The carrier is coated with the at least one color pigment and the matrix material. The coating is applied, for example, by vapor deposition under a high vacuum.

During deposition from the vapor phase, the at least one color pigment and the matrix material can be deposited on the carrier simultaneously (mixed vapor deposition, composite formation) or sequentially (layered composite formation).

According to a fourth general aspect of the invention, the said objective is solved by a method of inactivating viruses wherein the self-disinfecting antiviral filter material according to the invention or one of the embodiments is brought into contact with the viruses or a virus-containing material in the presence of visible light.

The filter material according to the invention and the air filter device provided with said filter material advantageously solve the above-mentioned objective by means of the antiviral, reactive effect of the color pigment, which changes (inactivates) the virus particles into a non-infectious state, in particular denatures or destroys them, on contact with virus particles which, for example, come from the air and/or from aerosols in the air onto the filter material and/or otherwise enter the filter material. The filter material according to the invention is therefore characterized by self-disinfection and disinfection of the air passing through it. The term “disinfection” refers to the transfer of all virus particles to the non-infectious state, or of such a large proportion of virus particles that the treated air is not infectious when inhaled.

The inorganic matrix material used according to the invention has particular advantages for fixing the at least one color pigment and performs three important tasks: It increases adhesion to the carrier, improves mechanical stability, and enhances the activity of the antiviral component. The increase in activity can be based, for example, on an increase in the efficiency of singlet oxygen formation by disaggregation of the at least one color pigment (disaggregation of a color pigment matrix), which consequently assumes a largely molecular photoactive state.

Complex syntheses for fixing the at least one color pigment and out-diffusion of the at least one color pigment can be avoided. The formation of a chemical linkage of the at least one color pigment with the carrier is possible, but not necessary. Restrictions to specific carriers can thus be avoided. In contrast to polymer embedding, inorganic matrix material allows an increase in singlet oxygen formation efficiency, since dye molecules contained in the inorganic matrix material can also come into contact with atmospheric oxygen.

The term “color pigment”, as used here, denotes a coloring agent, that is to say, a coloring substance (dye molecules), which absorbs light in the visible range of the electromagnetic spectrum and, in contrast to soluble dyes, comprises particles which are essentially insoluble in the respective liquid application medium or ambient milieu or under the conditions of use.

The color pigments with antiviral activity used according to the invention are preferably color pigments which can react upon exposure to light and in the presence of atmospheric oxygen (in the triplet state) to form singlet oxygen. The photochemical formation of singlet oxygen takes place particularly preferably upon exposure to visible light with a wavelength in the range from 400 nm to 750 nm. Singlet oxygen advantageously forms a strong oxidant with which the virus particles are denatured or destroyed.

Reactive singlet oxygen is known to be able to kill viruses (see A. Dewilde in “J. Photochem. Photobiol.” 36 (1996) 23). The formation of singlet oxygen thus enables continuous self-disinfection of the filter material according to the invention in air and in daylight or artificial light and disinfection of air passing through it.

The light is advantageously provided by ambient light when a breathing mask is worn. An air filter device provided with the filter material may also be configured to expose the filter material to ambient light and/or be provided with an illumination device, for example based on light-emitting diodes, which is adapted to expose the filter material to light.

In a preferred embodiment, the at least one color pigment comprises at least one compound from the group of phthalocyanines, this group comprising in particular unsubstituted phthalocya nine, substituted phthalocyanine derivatives and complexes, in particular metal complexes, thereof. Particularly preferred are unsubstituted phthalocyanine itself (C_{32H}H₁₈N₈) or metal-phthalocyanine complexes, selected in particular from the group comprising ZnPc, CIAIPc, FePc, NiPc, CoPc and CuPc (Pc here stands for phthalocyanine). The phthalocyanine compounds used according to the invention are typically water-insoluble and unsubstituted. However, it is also possible to use substituted phthalocyanines, which are preferably also water-insoluble. Advantageously, the color pigments used according to the invention are characterized by a particularly high yield of singlet oxygen formation.

In a particularly preferred embodiment, ZnPc or CIAIPc are used as metal phthalocyanines for the composite coating by high-vacuum evaporation, the layer thicknesses preferably varying between 20 nm and 500 nm.

The term “inorganic matrix material” generally refers to any inorganic compound that is at least partially transparent in the visible range of the electromagnetic spectrum.

According to a preferred embodiment, the inorganic matrix material is selected from the group comprising metal oxides (including silicon dioxide), and particularly preferably selected from the group comprising SiO₂, TiO₂, ZnO, MoO₃ and mixtures thereof (mixtures of at least two of these compounds). These matrix materials may be produced, deposited on the carrier and used in compact form or in nanoporous form, the nanoporous form being preferred for certain embodiments.

The composite coating of color pigment and matrix material is preferably at least one thin layer, in particular a layer with a thickness in the range of 10 nm to 1000 nm. The proportion of color pigment in the matrix material is typically in the range of 1 to 50% by volume, based on the matrix material.

The composite coating is disposed on the air-permeable carrier. The term “air-permeable carrier” is generally used to describe any material that accommodates the composite coating and has a structure, in particular pores or holes, through which air can flow.

Advantageously, the carrier remains air-permeable even when it ch as a layer, leaf or sheet, and/or a multi-part material, such as a granulate in a suitable holder or multiple layers, leaves or sheets.

Composites are preferably used as a coating on the carrier, these being deposited on the carrier by evaporation, in particular multihas received the composite coating, which is supported by the structure without sealing it, so that the filter material is air-permeable. The carrier may comprise a one-piece material, su source evaporation, of a phthalocyanine, in particular metal phthalocyanine, with at least one metal oxide applied simultaneously or sequentially under high vacuum, adhesion being advantageously improved by subsequent tempering. Tempering conditions may vary depending on the respective composite materials, but can easily be determined by a person skilled in the art using routine tests. For example, tempering can be carried out at a temperature in the range of 50° C. to 200 ° C. and for a period of time in the range of 10 minutes to 5 hours.

In a further particularly advantageous embodiment, SiO₂, TiO₂ or MoO₃ are used as metal oxides for the composite coating by high-vacuum evaporation. MoO₃ can advantageously be deposited simply by thermal evaporation, for example by resistance heating, in a high vacuum, whereas SiO₂ and TiO₂ can be evaporated at higher temperatures and are therefore typically deposited by electron beam evaporation (as described, for example, by Jager et al. in Adv. Mat. (1996) 93-97) or sputtering.

Another advantage of the invention is that a variety of materials are available and suitable to provide the air-permeable carrier. The carrier preferably comprises at least one layer of a layered or sheet-like material selected from the group consisting of woven textiles, non-woven textiles, air-permeable plastics, paper, in particular filter paper, cardboard, organic porous materials and inorganic porous materials.

The filter material according to the invention is preferably characterized in that the carrier with the composite coating is breathable, in particular suitable for breathing through the filter material, and/or in that the carrier with the composite coating is flexibly deformable.

The filter material according to the invention can be used in a freely selectable form as an antiviral active material in breathing masks or in antimicrobial air filters.

The filter material according to the invention is advantageously suitable for methods of destroying viruses wherein the filter material is brought into contact with viruses or virus-containing material, for example, air containing viruses or virus-containing material. If the color pigment with antiviral activity of the filter material according to the invention is a color pigment which reacts upon exposure to light and in the presence of oxygen to form singlet oxygen, contact in such a method accordingly takes place in the presence of light and oxygen.

Further details and advantages of the invention are described with reference to the accompanying drawings. They show schematically:

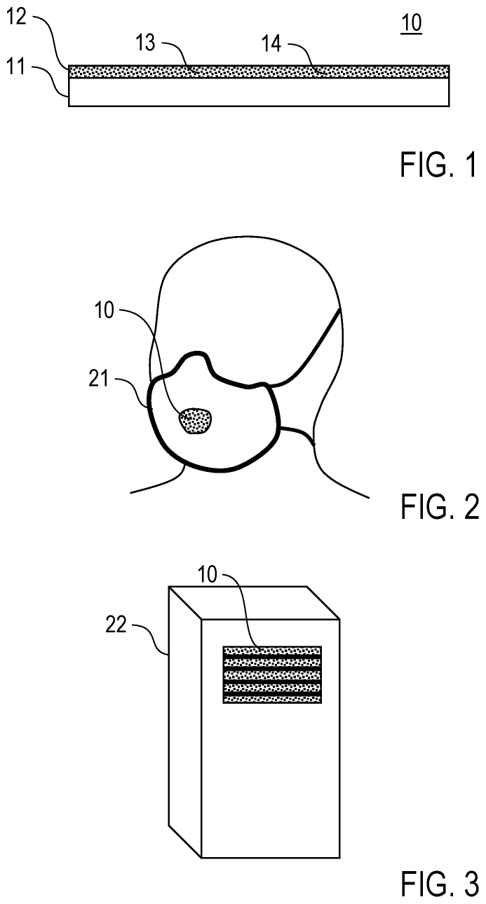

FIG. 1: a sectional view of an embodiment of the filter material according to the invention,

FIG. 2: an application of the filter material in a breathing mask, and

FIG. 3: an application of the filter material in an air purification device.

In a specific embodiment, a layer of the filter material 10 (see FIG. 1, not to scale) of a conventional breathing mask is provided as a carrier 11 onto which a composite coating 12 with the color pigment 13, for example ZnPc, and the matrix material 14, for example SiO₂, is applied in equal proportions by volume with a layer thickness of, for example, 300 nm. The filter material 10 is adapted for the specific application by cutting it to the size and shape of a filter receptacle of an air filter device, such as a breathing mask 21 (see FIG. 2), or in an air purification device 22 (see FIG. 3).

Viruses are inactivated when the air filter device is used, by the fact that air is drawn through the light-exposed filter material 10, is spread over the composite coating and passes through it. The air is transported, for example, by inhalation pressure on the breathing mask 21 or by a pumping apparatus of the air purification device 22.

The features of the invention disclosed in the foregoing description, the drawings and the claims may be significant, both individually and in combination or sub-combination, for the realization of the invention in its various embodiments.

Listing of claims:

1. A self-disinfecting antiviral filter material comprising
an air-permeable carrier, and
a composite coating bonded to the carrier and comprising at least one color pigment with antiviral activity and an inorganic matrix material.

2. The self-disinfecting material according to claim 1, wherein
the at least one color pigment is effective to react upon exposure to light and in a presence of oxygen to form singlet oxygen with antiviral activity.

3. The self-disenfecting antiviral filter material according to claim 2, wherein
the at least one color pigment is effective to react upon exposure to visible light in a wavelength range from 400 nm to 750 nm.

4. The self-disenfecting antiviral material according to claim 1, wherein
the at least one color pigment comprises at least one compound selected from the group consisting of phthalocyanines.

5. The self-disinfecting, antiviral filter material according to claim 1, wherein
the at least one color pigment is at least one compound selected from the group consisting of ZnPc, C1A1Pc, FePc, NiPc, CoPc, CuPc and C₃₂H₁₈N₈.

6. The self-disinfecting antiviral filter material according to claim 1, wherein
the matrix material is selected from the group consisting of metal oxides.

7. The self-disinfecting antiviral filter material according to claim 6, wherein
the matrix material is selected from the group consisting of SiO₂, TiO₂, ZnO, MoO₃ and mixtures thereof.

8. The self-disinfecting filter material according to claim 1, wherein
the composite coating comprises phthalocyanine pigments and at least one metal oxide that form a multi-layer or mixed layer deposited from a vapor phase.

9. The self-disinfecting antivira1 filter material according to claim 1, wherein
the carrier comprises at least one material selected from the group consisting of woven textiles, non-woven textiles, air-permeable plastics, paper, cardboard, organic porous materials and inorganic porous materials.

10. The self-disinfecting filter material according to the claim 1, wherein
the carrier with composite coating is breathable.

11. The self-disinfecting antiviral filter material according to claim 1, wherein
the carrier with composite coating is flexibly deformable.

12. An air filter device provided with the self-disinfecting antiviral filter material according to claim 1.

13. The air filter device according to claim 12, comprising a breathing mask

14. A method of manufacturing the self-disinfecting antiviral filter material according to claim 1, comprising the steps:
providing the air-permeable carrier, and
coating the carrier with at least one color pigment and an inorganic matrix material.

15. The method according to claim 14, wherein the coating of the carrier is realized by deposition of at least one of the color pigment and of the matrix material from a vapor phase under high vacuum.

16. The method according to claim 15, wherein the deposition of the color pigment and the deposition of the matrix material from the vapor phase are carried out simultaneously.

17. The method according to claim 15, wherein the deposition comprises a color pigment deposition followed by a matrix material deposition.

18. The method according to claim 14, wherein, after the deposition of the color pigment and of the matrix material to provide a deposited material, the deposited material is tempered.

19. A method of inactivating viruses, wherein the self-disinfecting antiviral filter material according to claim 1 is brought into contact with the viruses or a virus-containing material.

20. The method according to claim 19, wherein contact takes place in a presence of light and oxygen.

21. The air filter device according to claim 12, comprising an air filter of an air purification device.

22. The air filter device according to claim 12, comprising an air filter of an air purification device of an air-conditioning system.
